(19) United States
(12) Patent Application Publication
(10) Pub. No.: US 2020/0222074 A1
(43) Pub. Date: Jul. 16, 2020

(54) SPHINCTEROTOMES AND METHODS FOR USING SPHINCTEROTOMES

(71) Applicant: BOSTON SCIENTIFIC SCIMED, INC., Maple Grove, MN (US)

(72) Inventors: EVAN WILDER, Boston, MA (US); PAUL SMITH, Smithfield, RI (US); PETER L. DAYTON, Brookline, MA (US)

(73) Assignee: BOSTON SCIENTIFIC SCIMED, INC., Maple Grove, MN (US)

(21) Appl. No.: 16/742,539

(22) Filed: Jan. 14, 2020

Related U.S. Application Data
(60) Provisional application No. 62/792,689, filed on Jan. 15, 2019.

(51) Int. Cl. A61B 17/32

(52) U.S. Cl. CPC A61B 17/320016; A61B 2017/00867

(57) ABSTRACT

Sphincterotomes and methods for making and using sphincterotomes are disclosed. An example sphincterotome may include an elongate shaft having an outer surface and a distal end region. The sphincterotome may also include a sphincterotome wire assembly having a distal end coupled to the distal end region of the elongate shaft and a body portion extending along the outer surface of the elongate shaft. The sphincterotome wire assembly may be designed to shift the distal end region of the elongate shaft between a first configuration and a curved configuration. The body portion of the sphincterotome wire assembly may include a cutting region and a non-conductive region.

### CROSS-REFERENCE TO RELATED APPLICATIONS

This application claims the benefit of priority under 35 U.S.C. § 119 of U.S. Provisional Application No. 62/792,689 filed Jan. 15, 2019, the entire disclosure of which is hereby incorporated by reference.

### TECHNICAL FIELD

The present disclosure pertains to medical devices, and methods for manufacturing medical devices. More particularly, the present disclosure pertains to sphincterotomes.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A sphincterotome is disclosed. The sphincterotome, comprises: an elongate shaft having an articulation region, a distal end region, and an outer surface; wherein the articulation region is designed to allow the distal end region of the elongate shaft to shift between a first configuration and a curved configuration; and a sphincterotome wire assembly having a body portion extending along the outer surface of the elongate shaft; wherein the body portion of the sphincterotome wire assembly has substantially the same length when the elongate shaft is in either the first configuration or the curved configuration.

Alternatively or additionally to any of the embodiments above, the body portion of the sphincterotome wire assembly has a proximal end and a distal end.

Alternatively or additionally to any of the embodiments above, the articulation region extends proximally from the distal end of the body portion of the sphincterotome wire assembly.

Alternatively or additionally to any of the embodiments above, the articulation region extends distally from the proximal end of the body portion of the sphincterotome wire assembly.

Alternatively or additionally to any of the embodiments above, the articulation region extends proximally from the proximal end of the body portion of the sphincterotome wire assembly.

Alternatively or additionally to any of the embodiments above, the articulation region extends both distally and proximally from the proximal end of the body portion of the sphincterotome wire assembly.

Alternatively or additionally to any of the embodiments above, the sphincterotome wire assembly includes a distal anchor that is anchored to the elongate shaft.

Alternatively or additionally to any of the embodiments above, a distal end of the body portion of the sphincterotome wire assembly is disposed adjacent to the distal anchor.

Alternatively or additionally to any of the embodiments above, a second section of the sphincterotome wire assembly is coupled to the distal anchor and extends within a lumen formed in the elongate shaft.

Alternatively or additionally to any of the embodiments above, the sphincterotome wire assembly shifts the elongate shaft between the first configuration and the curved configuration.

Alternatively or additionally to any of the embodiments above, an articulation wire is disposed within the elongate shaft, and wherein the articulation wire shifts the elongate shaft between the first configuration and the curved configuration.

Alternatively or additionally to any of the embodiments above, the elongate shaft includes one or more slits along the articulation region.

Alternatively or additionally to any of the embodiments above, the elongate shaft includes one or more cutouts along the articulation region.

A sphincterotome is disclosed. The sphincterotome comprises: an elongate shaft having an articulation region, a distal end region, and an outer surface; wherein the articulation region is designed to allow the distal end region of the elongate shaft to shift between a first configuration, a first curved configuration, and a second curved configuration; and a sphincterotome wire assembly having a body portion extending along the outer surface of the elongate shaft; wherein the body portion of the sphincterotome wire assembly has substantially the same length when the elongate shaft is in either the first curved configuration or the second curved configuration.

Alternatively or additionally to any of the embodiments above, the body portion of the sphincterotome wire assembly has a first length when the elongate shaft is in the first configuration and a second length shorter than the first length when the elongate shaft is in the first curved configuration.

Alternatively or additionally to any of the embodiments above, the body portion of the sphincterotome wire assembly has substantially the same length when the elongate shaft is in either the first configuration or the first curved configuration.

Alternatively or additionally to any of the embodiments above, the body portion of the sphincterotome wire assembly has a distal end; and wherein the articulation region extends proximally from the distal end of the body portion of the sphincterotome wire assembly.

Alternatively or additionally to any of the embodiments above, the body portion of the sphincterotome wire assembly has a proximal end; and wherein the articulation region extends distally from the proximal end of the body portion of the sphincterotome wire assembly, the articulation region extends proximally from the proximal end of the body portion of the sphincterotome wire assembly, or both.

Alternatively or additionally to any of the embodiments above, the elongate shaft includes one or more slits, one or more cutouts, or both along the articulation region.

A method for curving a sphincterotome is disclosed. The method comprises: advancing a sphincterotome through a body lumen to a position adjacent to an area of interest; wherein the sphincterotome includes: an elongate shaft having an articulation region, a distal end region, and an outer surface, and a sphincterotome wire assembly having a body portion extending along the outer surface of the elongate shaft; applying a first proximal force to a proximal end region of the sphincterotome wire assembly to shift the elongate shaft from a first configuration to a first curved configuration; applying a second proximal force to the proximal end region of the sphincterotome wire assembly to shift the elongate shaft from the first curved configuration to a second curved configuration; and wherein the body portion of the sphincterotome wire assembly has substantially the same length when the elongate shaft is in either the first curved configuration or the second curved configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIGS. 1-3 are side views of a portion of an example sphincterotome.

FIGS. 4-6 are cross-sectional views of the sphincterotome shown in FIGS. 1-3.

FIG. 7 is a side view of a portion of an example sphincterotome.

FIG. 8 is a side view of a portion of an example sphincterotome.

FIG. 9 is a side view of a portion of an example sphincterotome.

FIG. 10 is a side view of a portion of an example sphincterotome.

FIG. 11 is a side view of a portion of an example sphincterotome.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Endoscopic retrograde cholangiopancreatography (ERCP) may be utilized to diagnose and treat various disorders of the pancreaticobiliary system. A clinician may use a sphincterotome to cannulate the papillary orifice. The sphincterotome may have a number of features including a cutting wire for performing a sphincterotomy. Typical sphincterotomes can be bend or bowed. When bowed, the sphincterotome may help to direct a guidewire (e.g. a guidewire extending through the sphincterotome). Bowing may occur by drawing the cutting wire into a proximal opening in the sphincterotome shaft. When do so, the length of the exposed cutting wire decreases as the degree of bowing increases. Disclosed herein are sphincterotomes that include structural features that allow the sphincterotome to curve or bow while having a reduced, minimal, or no impact on the length of the exposed cutting wire.

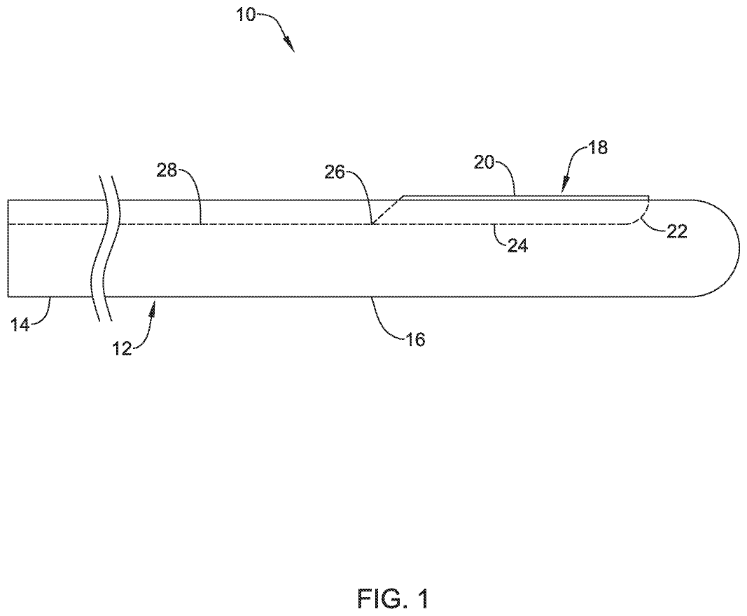

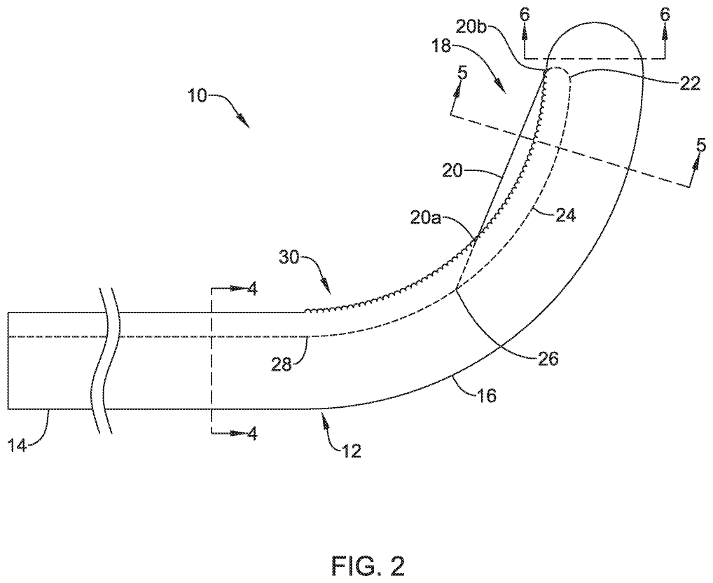

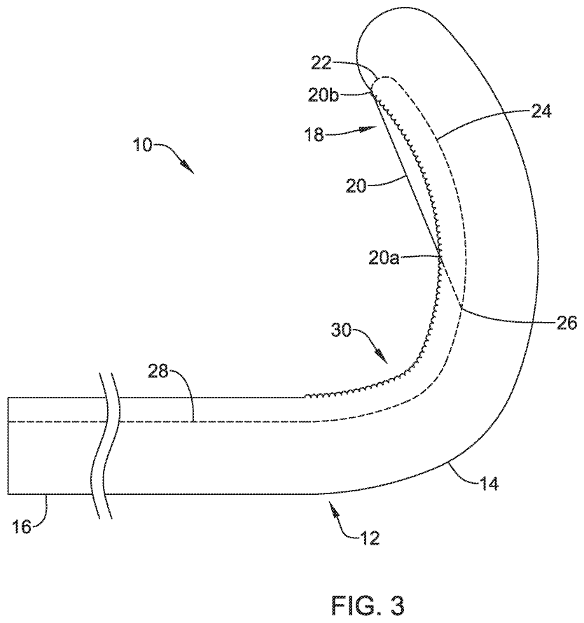

FIGS. 1-3 illustrates an example sphincterotome 10. The sphincterotome 10 may include an elongate shaft 12 having a proximal end region 14 and a distal end region 16. The sphincterotome 10 may include a sphincterotome wire assembly 18. The distal end region 16 of the elongate shaft 12 may be designed to shift between a first configuration (e.g., as depicted in FIG. 1), a first curved or bowed configuration (e.g., as depicted in FIG. 2), and a second curved or bowed configuration (e.g., as depicted in FIG. 3). For example, exerting a proximal force on one or more of the components of the sphincterotome wire assembly 18 may cause the elongate shaft 12 to shift between configurations. The sphincterotome wire assembly 18 may include a body portion 20 generally defined as the portion of the sphincterotome wire assembly 18 extending along the outer surface of the elongate shaft 12 and that may be described as extending diagonally akin to the string of a bow when the elongate shaft 12 is in the curved configurations (e.g., as depicted in FIGS. 2-3). The sphincterotome wire assembly 18 may also include a first or distal anchor/region 22, a second section 24, a junction region 26, and proximal wire section 28.

As schematically depicted in FIGS. 2-3, the elongate shaft 12 may include an articulation region 30. The articulation region 30, in general, is designed to allow the shaft 12 (e.g., the distal end region 16 of the shaft 12) to bend, curve, and/or otherwise take a different shape. In other words, the articulation region 30 allows the shaft 12 to shift between a first configuration (e.g., as depicted in FIG. 1), a first curved or bowed configuration (e.g., as depicted in FIG. 2), and a second curved or bowed configuration (e.g., as depicted in FIG. 3). In some instances, the articulation region 30 may function as a joint or hinge that allows for bending of the shaft 12.

In some instances, the articulation region 30 may include one or more structural features that allow the shaft 12 to bend/curve. For example, the articulation region 30 may include one or more cutouts or windows formed in the shaft 12. In some of these and in other instances, the articulation region 30 may include one or more slots, slits, scores, perforations, bellows, baffles, and/or the like. In some of these and in other instances, the articulation region 30 may be formed by using a lower durometer materials in the shaft 12, lower/differing durometer sheaths disposed along the articulation region 30, tapering and/or thinning of the wall of the shaft 12, a linkage formed in the shaft 12 (e.g., which may facilitate bending in one plane), and/or the like.

In some instances, the structural features of the articulation region 30 (e.g., the structural features that allows the shaft 12 to more easily bend/curve) may be disposed along the shaft 12 in a desirable manner. For example, the structural features of the articulation region 30 may be disposed along one side of the shaft 12, along opposite sides of the shaft 12, in a regular pattern along the shaft 12, in an irregular pattern along the shaft 12, etc. The length of the articulation region 30 may also vary. In at least some instances, the articulation region 30 may extend proximally from a proximal end 20a (e.g., where the proximal end 20a of the body portion 20 is disposed at a proximal opening in the shaft 12 where sphincterotome wire assembly 18 extends into the shaft 12) of the body portion 20. In some of these and in other instances, the articulation region 30 may extend distally from a proximal end 20a of the body portion 20. In some instances, the articulation region 30 may extend proximally from a distal end 20b (e.g., where the distal end 20b of the body portion 20 is disposed at a distal opening in the shaft 12 where sphincterotome wire assembly 18 extends into the shaft 12) of the body portion 20. These are just examples.

In at least some instances, shifting the shaft 12 between the first configuration, the first curved configuration, and/or the second curved configuration may include actuating the sphincterotome wire assembly 18. It can be appreciated that if a proximal force is applied to the proximal wire section 28 of the sphincterotome wire assembly 18 while the shaft 12 is in the first configuration, the shaft 12 may be pulled toward the first and/or second curved configuration. In shafts that do not include an articulation region 30, it can be appreciated that the length of the body portion 20 would become shorter as the shaft 12 bends. Because the body portion 20 may be used to cut and/or help to cannulate during an intervention, shortening the length of the “cutting” wire (e.g., the body portion 20) may impact the intervention. It may be desirable to keep the length of the body portion 20 substantially constant.

As indicated above, the articulation region 30 allows the shaft 12 to more easily bend/curve. Because of this, when a proximal force is applied to the proximal wire section 28 of the sphincterotome wire assembly 18 while the shaft 12 is in the first configuration, the shaft 12 may be pulled/bent toward the first and/or second curved configuration by bending at/along the articulation region 30. Because the articulation region 30 may more freely bend, a clinician may be able to bend the shaft 12 without having to shorten the length of the body portion 20. For example, the body portion 20 may have a length when the shaft 12 is in the first configuration. When the shaft 12 in the first curved configuration, the body portion 20 may have substantially the same length (e.g., the length of the body portion 20 when the shaft 12 is in the first configuration is substantially the same as the length of the body portion when the shaft 12 is in the first curved configuration). This may be because the sphincterotome wire assembly 18 need not be shortened when further curving the shaft 12 as the articulation region 30 allows the shaft 12 to bend without having to cinch up or shorten the sphincterotome wire assembly 18 (e.g., and/or cinch up or shorten the body portion 20).

When the shaft 12 is in the second curved configuration, the body portion 20 may have substantially the same length as when in the first configuration (e.g., the length of the body portion 20 when the shaft 12 is in the first configuration is substantially the same as the length of the body portion when the shaft 12 is in the second curved configuration) and/or the first curved configuration (e.g., the length of the body portion 20 when the shaft 12 is in the first curved configuration is substantially the same as the length of the body portion when the shaft 12 is in the second curved configuration). Again, this may be because the sphincterotome wire assembly 18 need not be shortened when further curving the shaft 12 as the articulation region 30 allows the shaft 12 to bend without having to cinch up or shorten the sphincterotome wire assembly 18 (e.g., and/or cinch up or shorten the body portion 20).

In some instances, the length of the body portion 20 may be slightly reduced when the shaft 12 shift from the first configuration to the first curved configuration. For example, the length of the body portion 20 may be reduced by about 10% or less, or about 5% or less, or about 2% or less. This may be because the sphincterotome wire assembly 18 may be shortened when initially curving the shaft 12. When further shifting the shaft 12 from the first curved configuration to the second curved configuration, the body portion 20 may maintain a substantially constant length. This may be because the sphincterotome wire assembly 18 need not be shortened when further curving the shaft 12 as the articulation region 30 allows the shaft 12 to bend without having to cinch up or shorten the sphincterotome wire assembly 18 (e.g., and/or cinch up or shorten the body portion 20).

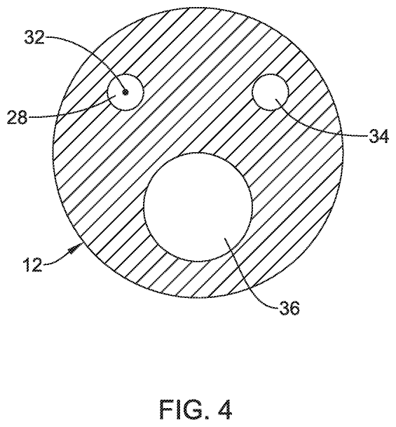

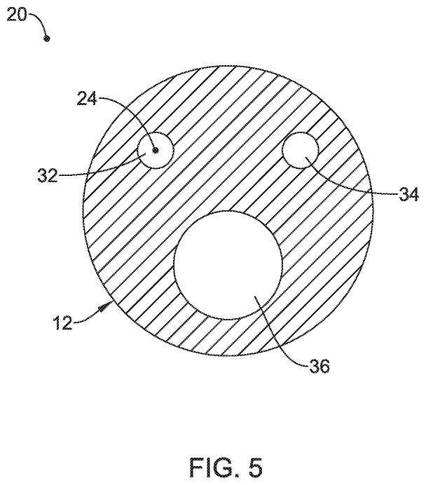

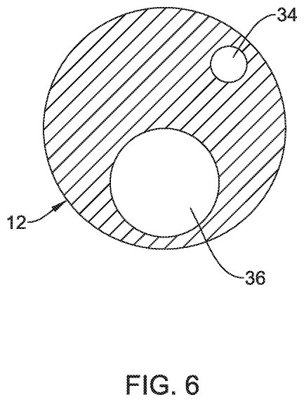

FIGS. 4-6 are cross-sectional views taken at various locations along the shaft 12. Here it can be seen that the shaft 12 may include a number of different lumens. For example, the shaft 12 may include a first lumen 32, a second lumen 34, and a third lumen 36. In this example, the proximal wire section 28 may extend through the first lumen 32. The second lumen 34 may be used for infusing a fluid such as a contrast media. The third lumen 36 may be a guidewire lumen.

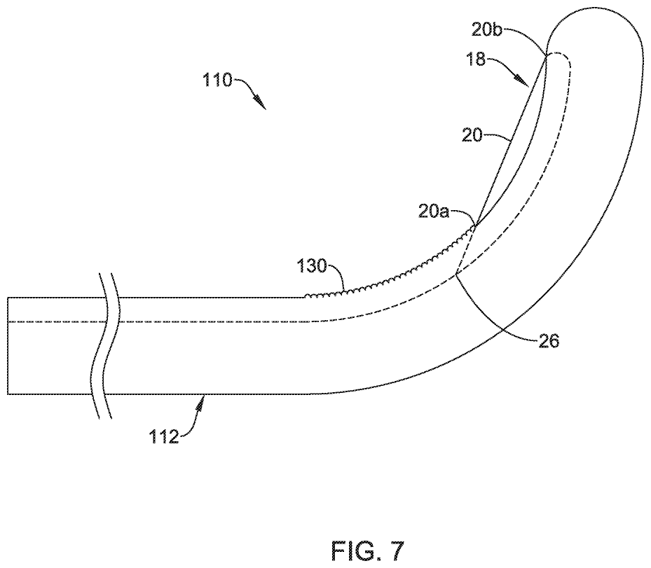

FIG. 7 illustrates another example sphincterotome 110 that may be similar in form and function to other sphincterotomes disclosed herein. The sphincterotome 110 may include a shaft 112 that includes an articulation region 130. In this example, the articulation region 130 extends proximally from the proximal end 20a of the body portion 20. In this example, the articulation region 130 does not extend distally of the proximal end 20a of the body portion 20.

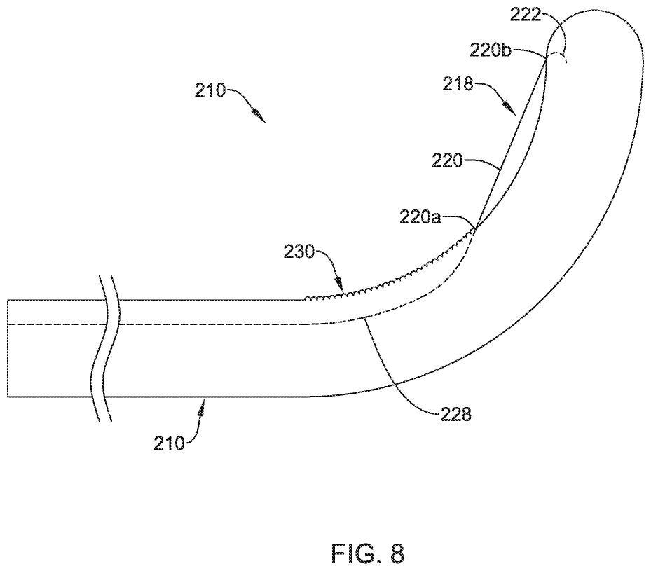

FIG. 8 illustrates another example sphincterotome 210 that may be similar in form and function to other sphincterotomes disclosed herein. The sphincterotome 210 includes a shaft 212 having an articulation region 230. The sphincterotome 210 may include a sphincterotome wire assembly 218 including a body portion 220. The body portion 220 may include a proximal end 220a and a distal end 220b. In this example, the articulation region 230 extends proximally from the proximal end 220a of the body portion 220. The sphincterotome wire assembly 218 may also include a first or distal anchor or distal anchor region 222 and a proximal wire section 228. The distal anchor region 222 may help the body portion 220 maintain a substantially constant length when the elongate shaft 212 is in either the first configuration or the curved configuration. In this example, the sphincterotome wire assembly 218 may be free of a second section (e.g., similar to the second section 24).

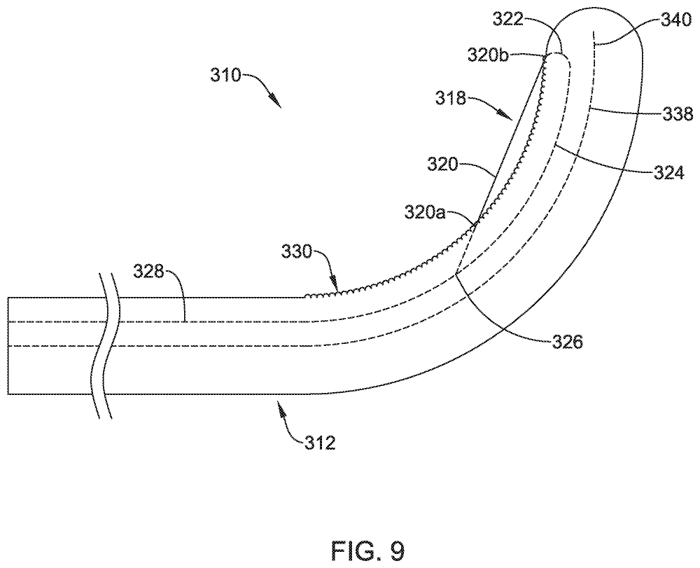

FIG. 9 illustrates another example sphincterotome 310 that may be similar in form and function to other sphincterotomes disclosed herein. The sphincterotome 310 includes a shaft 312 having an articulation region 330. The sphincterotome 310 may include a sphincterotome wire assembly 318 including a body portion 320. The body portion 320 may include a proximal end 320a and a distal end 320b. In this example, the articulation region 330 extends proximally and distally from the proximal end 320a of the body portion 320. In addition, the articulation region 330 extends proximally from the distal end 320b of the body portion 320. The sphincterotome wire assembly 318 may also include a first or distal anchor/region 322, a second section 324, a junction region 326, and proximal wire section 328.

In this example, shifting the shaft 312 between the first configuration, the first curved configuration, and/or the second curved configuration may be accomplished without needing to pull on the sphincterotome wire assembly 318. Instead, the sphincterotome 310 may include an articulation wire 338 disposed within the shaft 312. The articulation wire 338 may include a first or distal anchor/region 340. The articulation wire 338 may be pulled (e.g., a proximally-directed force may be applied to the articulation wire 338) to shift the shaft 312 between configurations.

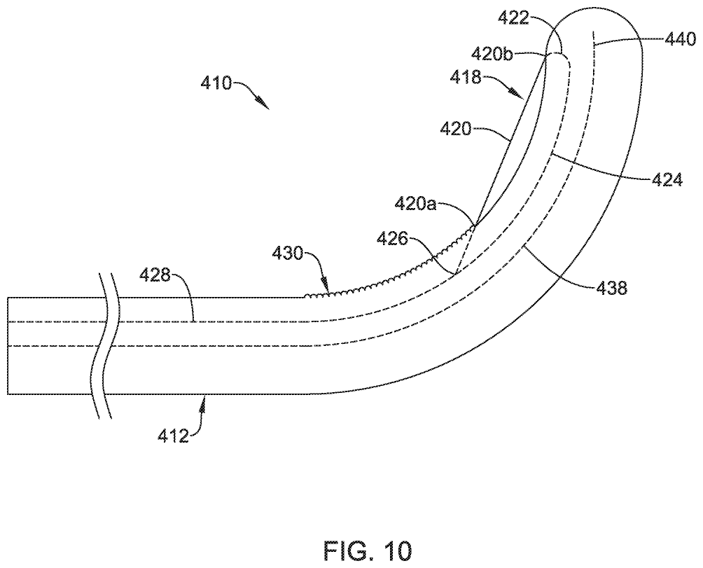

FIG. 10 illustrates another example sphincterotome 410 that may be similar in form and function to other sphincterotomes disclosed herein. The sphincterotome 410 includes a shaft 412 having an articulation region 430. The sphincterotome 410 may include a sphincterotome wire assembly 418 including a body portion 420. The body portion 420 may include a proximal end 420a and a distal end 420b. In this example, the articulation region 430 extends proximally from the proximal end 420a of the body portion 420. The sphincterotome wire assembly 418 may also include a first or distal anchor/region 422, a second section 424, a junction region 426, and proximal wire section 428. An articulation wire 438 may be disposed within the shaft 412. The articulation wire 438 may include a first or distal anchor/region 440. The articulation wire 438 may be pulled (e.g., a proximally-directed force may be applied to the articulation wire 438) to shift the shaft 412 between configurations.

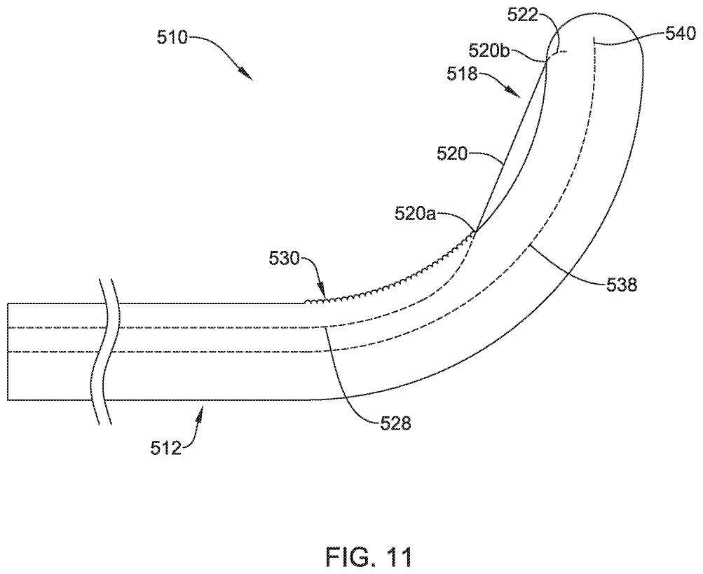

FIG. 11 illustrates another example sphincterotome 510 that may be similar in form and function to other sphincterotomes disclosed herein. The sphincterotome 510 includes a shaft 512 having an articulation region 530. The sphincterotome 510 may include a sphincterotome wire assembly 518 including a body portion 520. The body portion 520 may include a proximal end 520a and a distal end 520b. In this example, the articulation region 530 extends proximally from the proximal end 520a of the body portion 520. The sphincterotome wire assembly 518 may also include a first or distal anchor/region 522 and proximal wire section 528. An articulation wire 538 may be disposed within the shaft 512. The articulation wire 538 may include a first or distal anchor/region 540. The articulation wire 538 may be pulled (e.g., a proximally-directed force may be applied to the articulation wire 538) to shift the shaft 512 between configurations.

The materials that can be used for the various components of the sphincterotomes disclosed herein may include those commonly associated with medical devices. For simplicity purposes, the following discussion makes reference to the shaft 12. However, this is not intended to limit the devices and methods described herein, as the discussion may be applied to other devices and/or components of devices disclosed herein.

The shaft 12 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), perfluoroalkoxy alkane (PFA), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the sphincterotome 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the sphincterotome 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the sphincterotome 10 to achieve the same result.

In some embodiments, a degree of Magnetic Resonance Imaging (MRI) compatibility is imparted into the sphincterotome 10. For example, the sphincterotome 10, or portions thereof, may be made of a material that does not substantially distort the image and create substantial artifacts (e.g., gaps in the image). Certain ferromagnetic materials, for example, may not be suitable because they may create artifacts in an MRI image. The sphincterotome 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A sphincterotome, comprising:
an elongate shaft having an articulation region, a distal end region, and an outer surface;
wherein the articulation region is designed to allow the distal end region of the elongate shaft to shift between a first configuration and a curved configuration; and
a sphincterotome wire assembly having a body portion extending along the outer surface of the elongate shaft;
wherein the body portion of the sphincterotome wire assembly has substantially the same length when the elongate shaft is in either the first configuration or the curved configuration.

2. The sphincterotome of claim 1, wherein the body portion of the sphincterotome wire assembly has a proximal end and a distal end.

3. The sphincterotome of claim 2, wherein the articulation region extends proximally from the distal end of the body portion of the sphincterotome wire assembly.

4. The sphincterotome of claim 2, wherein the articulation region extends distally from the proximal end of the body portion of the sphincterotome wire assembly.

5. The sphincterotome of claim 2, wherein the articulation region extends proximally from the proximal end of the body portion of the sphincterotome wire assembly.

6. The sphincterotome of claim 2, wherein the articulation region extends both distally and proximally from the proximal end of the body portion of the sphincterotome wire assembly.

7. The sphincterotome of claim 1, wherein the sphincterotome wire assembly includes a distal anchor that is anchored to the elongate shaft.

8. The sphincterotome of claim 7, wherein a distal end of the body portion of the sphincterotome wire assembly is disposed adjacent to the distal anchor.

9. The sphincterotome of claim 7, wherein a second section of the sphincterotome wire assembly is coupled to the distal anchor and extends within a lumen formed in the elongate shaft.

10. The sphincterotome of claim 1, wherein the sphincterotome wire assembly shifts the elongate shaft between the first configuration and the curved configuration.

11. The sphincterotome of claim 1, wherein an articulation wire is disposed within the elongate shaft, and wherein the articulation wire shifts the elongate shaft between the first configuration and the curved configuration.

12. The sphincterotome of claim 1, wherein the elongate shaft includes one or more slits along the articulation region.

13. The sphincterotome of claim 1, wherein the elongate shaft includes one or more cutouts along the articulation region.

14. A sphincterotome, comprising:
an elongate shaft having an articulation region, a distal end region, and an outer surface;
wherein the articulation region is designed to allow the distal end region of the elongate shaft to shift between a first configuration, a first curved configuration, and a second curved configuration; and
a sphincterotome wire assembly having a body portion extending along the outer surface of the elongate shaft;
wherein the body portion of the sphincterotome wire assembly has substantially the same length when the elongate shaft is in either the first curved configuration or the second curved configuration.

15. The sphincterotome of claim 14, wherein the body portion of the sphincterotome wire assembly has a first length when the elongate shaft is in the first configuration and a second length shorter than the first length when the elongate shaft is in the first curved configuration.

16. The sphincterotome of claim 14, wherein the body portion of the sphincterotome wire assembly has substantially the same length when the elongate shaft is in either the first configuration or the first curved configuration.

17. The sphincterotome of claim 14, wherein the body portion of the sphincterotome wire assembly has a distal end; and
wherein the articulation region extends proximally from the distal end of the body portion of the sphincterotome wire assembly.

18. The sphincterotome of claim 14, wherein the body portion of the sphincterotome wire assembly has a proximal end; and
wherein the articulation region extends distally from the proximal end of the body portion of the sphincterotome wire assembly, the articulation region extends proximally from the proximal end of the body portion of the sphincterotome wire assembly, or both.

19. The sphincterotome of claim 14, wherein the elongate shaft includes one or more slits, one or more cutouts, or both along the articulation region.

20. A method for curving a sphincterotome, the method comprising:
advancing a sphincterotome through a body lumen to a position adjacent to an area of interest;
wherein the sphincterotome includes:
an elongate shaft having an articulation region, a distal end region, and an outer surface, and
a sphincterotome wire assembly having a body portion extending along the outer surface of the elongate shaft;
applying a first proximal force to a proximal end region of the sphincterotome wire assembly to shift the elongate shaft from a first configuration to a first curved configuration;
applying a second proximal force to the proximal end region of the sphincterotome wire assembly to shift the elongate shaft from the first curved configuration to a second curved configuration; and
wherein the body portion of the sphincterotome wire assembly has substantially the same length when the elongate shaft is in either the first curved configuration or the second curved configuration.
